(19) United States
(12) Patent Application Publication
(10) Pub. No.: US 2007/0190036 A1
(43) Pub. Date: Aug. 16, 2007

(54) Microencapsulation of pancreatic islets within a thin coat of glucagon-like peptide-1 functionalized poly(ethylene glycol)

(76) Inventors: Seda Kizilel, Chicago, IL (US); Jason L. Wyman, Chicago, IL (US); Milan Mrksich, Chicago, IL (US); Sidney R. Nagel, Chicago, IL (US); Marc R. Garfinkel, Chicago, IL (US)

Correspondence Address: BRINKS HOFER GILSON & LIONE, P.O. BOX 10395, CHICAGO, IL, 60610, US

(21) Appl. No.: 11/649,356

(22) Filed: Jan. 3, 2007

Related U.S. Application Data
(60) Provisional application No. 60/756,026, filed on Jan. 4, 2006.

(51) Int. Cl. A61K 35/39

(52) U.S. Cl. 424/93.7; 424/78.27

(57) ABSTRACT

Microencapsulation of pancreatic islets within a thin poly(ethylene glycol) (PEG) coat is provided. The PEG coat is functionalized with an insulinotropic agent, glucagon-like peptide 1 (GLP-1). The encapsulated pancreatic islets may be used for the treatment of type I diabetes.

### RELATED APPLICATIONS

The present patent document is a Utility Patent Application claiming priority under 35 U.S.C. §119 to Provisional Patent Application Ser. No. 60/756,026 filed Jan. 4, 2006, which is hereby incorporated by reference.

### FEDERALLY SPONSORED RESEARCH OR DEVELOPMENT

This invention was made with government support under DMR-0213745 and DMR-0352777 awarded by the National Science Foundation. The government has certain rights in the invention.

### BACKGROUND

1. Technical Field

The present invention relates to the microencapsulation of pancreatic islets within a thin poly(ethylene glycol) (PEG) coat. The invention includes functionalization of the PEG coat with an insulinotropic agent, glucagon-like peptide 1 (GLP-1). Pancreatic islets encapsulated according to the invention may be used for the treatment of type I diabetes.

2. Background Information

Microencapsulation, in which transplanted tissue is separated from the recipient's immune system by a semipermeable membrane has been investigated to overcome problems in whole organ and isolated human islet transplantation. For example, U.S. patent application Ser. No. 10/992,615 filed Jan. 18, 2005 and assigned to The University of Chicago, the entire teaching of which is incorporated herein by reference, teaches a selective withdrawal technique for micro-encapsulation of pancreatic islets. Prior microencapsulation techniques have been based primarily on alginate chemistry. These coatings typically have thicknesses of hundreds of microns. Such thick coats generally impair the diffusion of essential molecules such as oxygen and nutrients through the polymer layer. The technique provided by U.S. patent application Ser. No. 10/992,615 generates much thinner coats, having a mean thickness of about 20.5 μm. Unfortunately, even with the improvements described in the above mentioned patent application, obstacles remain in the path of developing effective diabetes treatments using microencapsulated pancreatic islets. For example, the scarcity of human pancreas or islet sources and the large number of islets required for effective treatment effectively limit the potential for such treatments. Another obstacle to the transplantation of islets is the need to suppress the immune system of the recipient to prevent the rejection of transplanted cells. Microencapsulation may help overcome such obstacles to islet transplantation, thus enabling the transplantation of microencapsulated pancreatic islets to be an effective weapon in the fight against diabetes.

### BRIEF SUMMARY

The present invention relates to the microencapsulation of pancreatic islets within a thin poly(ethylene glycol) (PEG) coat. The invention includes functionalization of the PEG coat with an insulinotropic agent, glucagon-like peptide 1 (GLP-1). Pancreatic islets encapsulated according to the invention may be used for the treatment of type I diabetes.

According to an embodiment of the invention, a functionalized coating for encapsulating pancreatic islets is formed of a thin poly(ethylene glycol) (PEG) hydrogel. The PEG hydrogel includes crosslinked networks of PEG diacrylate. An insulinotropic agent is incorporated into the crosslinked networks of the PEG diacrylate. The insulinotropic agent incorporated into the PEG diacrylate may comprise glucagon-like peptide-1 (GLP-1).

According to another embodiment, biological tissue is prepared for implantation into a patient. The tissue includes one or more donor cells. A coating encapsulates the one or more donor cells. The coating is functionalized with glucagon-like-peptide-1 (GLP-1). According to an embodiment, the donor |tissue|_{[JLW1]} may comprise pancreatic islets. The functionalized coating may be formed of a poly(ethylene glycol) (PEG) hydrogel.

Finally, another embodiment provides a method of coating biological material for implantation into a patient. The method calls for creating |a polymeric precursor |_{[JLW2]}solution that includes a peptide. Biological material such as pancreatic islets are added to the precursor solution. The biological material is encapsulated within the precursor solution by selectively withdrawing the biological material within the precursor solution. The encapsulating solution is photocrosslinked to form a hydrogel network incorporating the peptide. The precursor solution may comprise a poly(ethylene glycol) (PEG) diacrylate precursor solution. The peptide included in the pre-polymer solution may comprise glucagon-like-peptide-1. The method may further include dissolving the peptide in an aqueous sodium bi-carbonate buffer solution and separately dissolving acrylate-PEG-NHS in an aqueous sodium bi-carbonate buffer solution. The acrylate-PEG-NHS solution may be added to the peptide solution, and the combined solutions may be allowed to react to form a PEG-peptide conjugate.

Other systems, methods, features, and advantages of the invention will be, or will become, apparent to one with skill in the art upon examination of the following figures and detailed description. It is intended that all such additional systems, methods, features, and advantages be included within this description, be within the scope of the invention, and be protected by the following claims.

### BRIEF DESCRIPTION OF THE DRAWINGS

FIG. 1 is a diagram illustrating the proposed intracellular mechanism for the observed insulinotropic effects of GLP-1 on pancreatic β cells;

FIG. 2 is a diagram showing GLP-1 incorporated into a network of PEG diacrylate hydrogel.

FIG. 3 is a diagram showing the synthesis of acrylated peptide through the reaction of the peptide with acryloyl-PEG-N-hydroxysuccininimide (acryloyl-PEG-NHS).

FIG. 4 is mass spectrometer plots of PEG-NHS, GLP-1, and PEG-peptide conjugate.

FIG. 5 is a dynamic insulin secretion profile demonstrating a 44% increase in glucose-stimulated insulin secretion of GLP-1/PEG-encapsulated islets vs. naked islets or PEG-encapsulated islets without GLP-1.

FIG. 6 is a diagram showing the formation of an acrylate peptide from an N-hydroxy succinimidyl activated ester of acrylic acid.

### DETAILED DESCRIPTION OF THE DRAWINGS AND THE PRESENTLY PREFERRED EMBODIMENTS

In an embodiment of the invention insulinotropic agents are incorporated into PEG hydrogel encapsulating pancreatic islets. The presence of this peptide within the PEG coating can reduce the number of islets needed to normalize blood glucose of a diabetic patient by improving the insulin secretion ability of the microencapsulated islets.

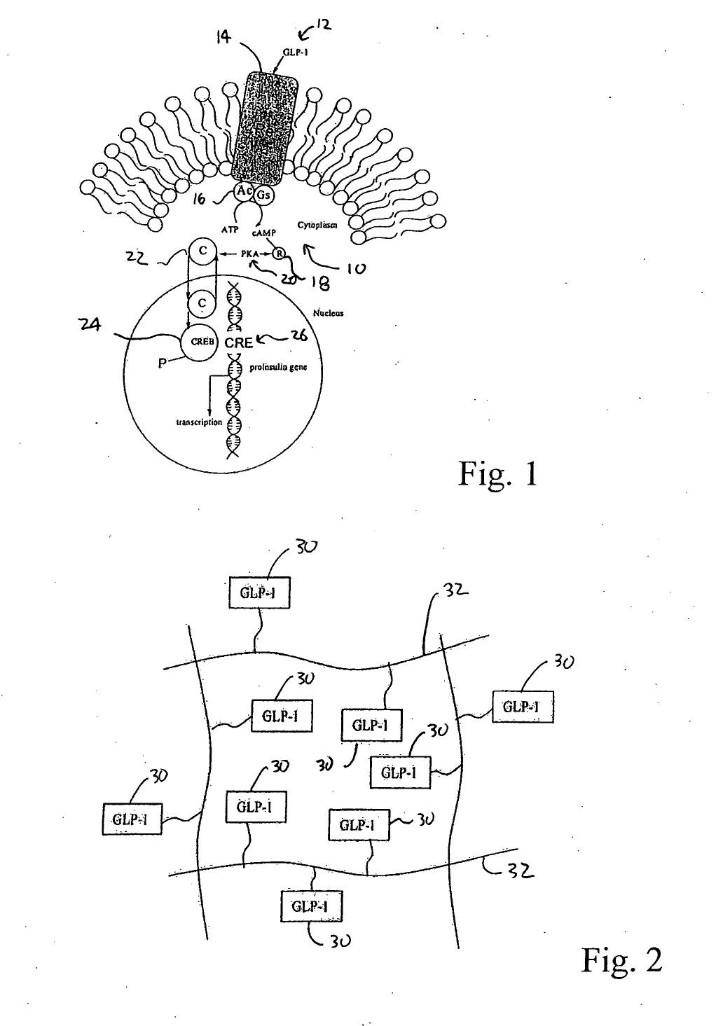

The insulinotropic hormone, also known as glucagon-like peptide 1, or GLP-1, is produced by intestinal L cells upon food intake. GLP-1 enhances insulin release from pancreatic β-cells in the pancreas in a glucose dependent manner. GLP-1 has a number of effects on the regulation of pancreatic β-cell mass, including the stimulation of insulin biosynthesis, the restoration of glucose sensitivity to the pancreatic islets, and stimulation of the growth and replication of pancreatic β-cells. FIG. 1 illustrates the proposed intracellular mechanism for the observed insulinotropic effects of GLP-1 on pancreatic β-cells. The process is mediated by activation of the cAMP-signaling pathway. The binding of GLP-1 12 to its receptor (Re) 14 activates adenylate cyclase (Ac) 16. This results in the formation of the cAMP signaling pathway 10. The Binding of cAMP to the regulatory (R) sub-unit 18 of PKA 20 results in the release of the active catalytic C sub-unit 22. This is followed by translocation of active kinase to the nucleus, and phosphorylation and activation of the nuclear transcriptional activator CREB 24. The nuclear transcriptional activator CREB 24 is bound to the CRE 26 and located in the promoter of the proinsulin gene. Transcription of the proinsulin gene is stimulated as a result of this cascade of signaling events. Insulin biosynthesis is increased to replete the stores of insulin secreted in response to glucose.

Despite the great therapeutic potential of GLP-1 to treat diabetes, it is quite unstable in vivo. GLP-1 has a half life of less than 2 minutes when administered intravenously. The short half life is due to the N-terminal cleavage of the first two amino acids, His8-Ala9 by the enzyme dipeptidyl peptidase IV (DPP-IV). Until recently, the main effort to improve the therapeutic level of GLP-1 has been the development of enzyme resistant GLP-1 analogues created by substituting one or two of the amino acids of the peptide. However, despite promising results, optimizing enzyme resistance and biological potency in vivo still remains an ongoing challenge. In order to improve clinical efficiency, the chemical conjugation of GLP-1 to various substances, such as fatty acid acylation, albumin conjugation, or PEGylation has been investigated. One such study involved conjugation of the GLP-1 peptide to PEG through amine terminus and lysine residues located on the 26th and 34th locations. The other study involved the conjugation of the GLP-1 peptide to a very large polymer and incorporating the conjugate with alginate to encapsulate islets. Even though the research has increased the half-life of GLP-1 and decreased its clearance, the use of GLP-1 as an anti-diabetic agent still requires frequent administration at relatively high doses.

According to an embodiment of the invention, the insulinotropic agent GLP-1 is incorporated into the PEG hydrogel capsule coating pancreatic islets. The presence of the GLP-1 peptide within the coating can reduce the number of islets needed to normalize blood glucose of a diabetic patient by improving the insulin secretion ability of the microencapsulated islets.

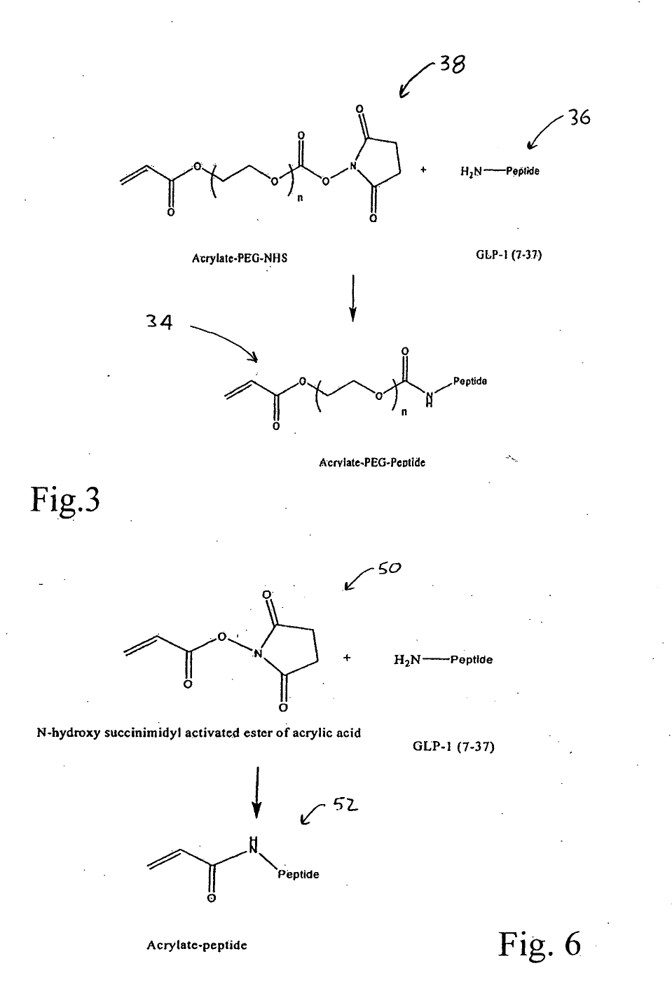

An embodiment of the invention incorporates GLP-1 into the PEG hydrogel network by chemical means, as shown in FIG. 2. The PEG coats are functionalized with the GLP-1 peptide 30. The GLP-1 peptide 30 is incorporated into the crosslinked networks of PEG diacrylate hydrogel 32. This is achieved by functionalizing the amine terminus of the peptide with an acrylate moiety. This enables the peptide to copolymerize rapidly with PEG diacrylate upon photoinitiation. Acrylated peptide 34 has been successfully synthesized as a result of the reaction of the peptide 36 with acryloyl-PEG-N-hydroxysuccinimide (acryloyl-PEG-NHS) ester 38 as shown in FIG. 3.

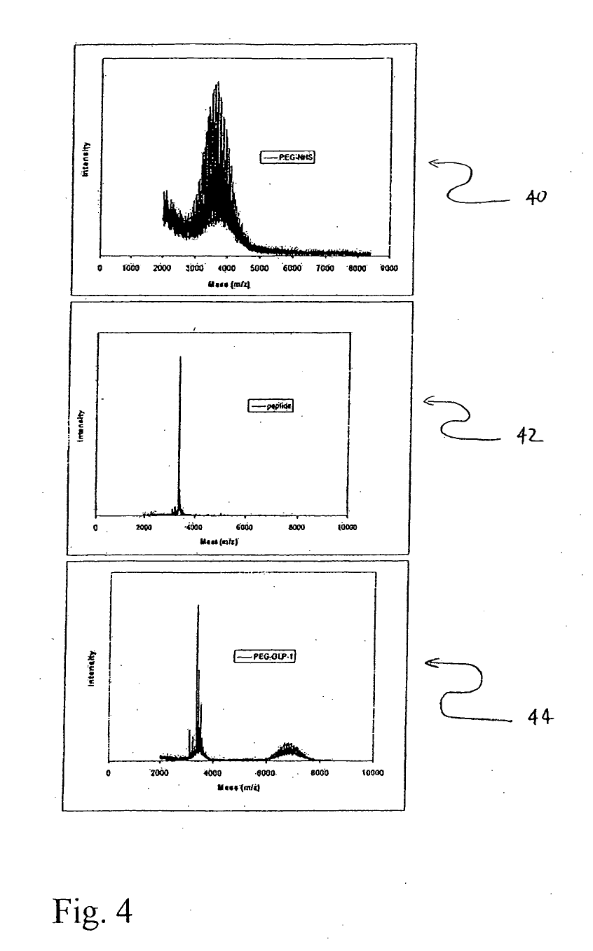

The chemical conjugation between acryloyl-PEG-NHS 38 and GLP-1 (7-37) 36 has been confirmed by mass spectrometry. FIG. 4 displays the mass spectral characterization of: a) PEG-NHS that has a molecular weight of 3400 40; b) GLP-1 (7-37) 42 that has a molecular weight of 3355.67; and c) PEG-peptide conjugate 44 which has a molecular weight around 6800. The area under the curves suggests that the GLP-1 content within the conjugate is about 10% (w/w).

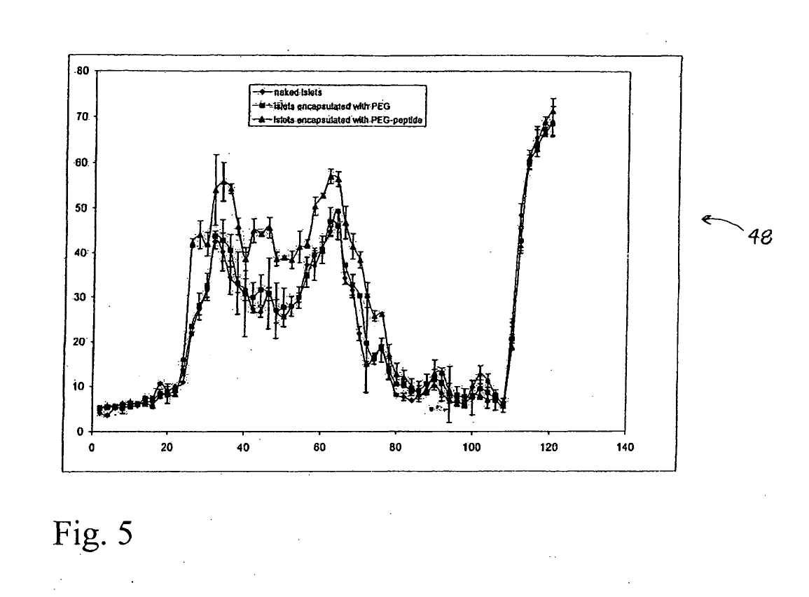

FIG. 5 displays the dynamic insulin secretion profile 48 of pancreatic islets, demonstrating a 44% increase in glucose-stimulated insulin secretion of GLP-1/PEG-encapsulated islets vs. naked islets or PEG encapsulated islets without GLP-1. The method for determining the profile was as follows. Fluid containing glucose was made to flow through three separate chambers. A first chamber contained 10 unencapsulated islets. The second chamber contained 10 islets encapsulated with just PEG. The last chamber contained 10 islets encapsulated with GLP-1 functionalized PEG. The glucose concentration within the fluid was strictly controlled to measure the amount of insulin produced by the islets in each chamber. A low initial glucose concentration of 3.3 mM simulated sub-normal physiological blood glucose levels. This resulted in the measurement of low insulin levels downstream. After 20 minutes the glucose concentration was increased to a stimulatory level of 16.7 mM. This level resulted in a rapid peak in the release of insulin, which then oscillated around an elevated baseline. After 40 minutes of high glucose levels the initial low glucose concentration of 3.3 mM was restored and insulin release decreased to its initial levels. Finally, potassium chloride was used to depolarize the membranes of the insulin-containing beta cells within the islets. This caused the islets to vigorously secrete the remaining stored insulin. The response of the islets encapsulated with PEG (square symbols) and unencapsulated (diamond symbols) islets were statistically indistinguishable from each other. However, based on an area under the curve analysis, the response of islets (triangle symbols) encapsulated with peptide functionalized coats, was 44% higher in insulin secretion than the two other sets during the high glucose perfusion.

### Acrylate-PEG Peptide Synthesis And Characterization

The following is a description of the process used to prepare the acrylate-PEG Peptide synthesis.

1. The peptide is dissolved to a final aqueous concentration of 1 mg/mL in 50 mM of sodium bicarbonate buffer, pH 8.2.

2. Acrylate-PEG-NHS is separately dissolved in 50 mM of bicarbonate buffer such that the final molar ratio of Acrylate-PEG-NHS to peptide molar ratio is 2. PEG solution (200 μl) is added dropwise to the peptide solution (1 mL). The PEG solution and the peptide solution are reacted at room temperature for 2 hours, and lyophilized (freeze-dried).

3. Reactions may be conducted at molar ratios of 5, 10, and 25 succinimidyl ester to a peptide for 2, 6, and 24 hours to maximize the conversion of peptide to the desired product and minimize side reactions (Scheme 1).

4. To minimize the production of acryloyl-PEG-OH, the product of the competing hydrolysis reaction, a low reactant ratio of 2 may be chosen. Alternatively, this product may be removed from acryloyl-PEG-peptide.

5. Acrylated and unacrylated peptides may be separated from the reaction byproducts acryloyl-PEG-OH, unreacted peptide and hydroxysuccinimide, by dialysis (MWCO 3500) against de-ionized water for 48 h against periodic bath changes.

6. Peptide content in PEG-peptide conjugate is determined using UV spectrophotometry. Acrylated peptide can be identified by the increase in absorption of the original peptide at 280 nm, where acrylate bonds absorb strongly.

FIG. 3 shows a schematic representation of acryloyl-PEG-peptide. FIG. 6 shows a schematic representation of acrylate-peptide formation.

### Peptide Solution

We begin with a 50 mM sodium bicarbonate buffer solution (NaHCO₃, MW=84.01 g/mole). In order to prepare a 20 mL peptide solution, 84 mg of NaHCO₃ is needed. The Final peptide concentration should be 1 mg/mL. Therefore, 20 mg of peptide should be added to 50 mM NaHCO₃ buffer. (Peptide MW=3355.67 g/mole)

| Total Volume (mL) | 50 mM (mg) | |
|---|---|---|
| of the solution | NaHCO₃ | Peptide (mg) |
| 10 | 42 | 10 |
| 20 | 84 | 20 |
| 40 | 168 | 40 |
| 80 | 336 | 80 |

### Acryloyl-PEG-NHS Solution (APN)

Again, we begin a 50 mM sodium bicarbonate buffer solution (NaHCO₃, MW=84.01 g/mole). In order to prepare a 4 mL APN solution, 16.8 mg of NaHCO₃ is required. The final APN concentration should be 10 mg/mL. Therefore 40 mg of APN should be added to the 50 mM NaHCO₃ buffer. (APN MW=3400 g/mole.)

| Total Volume (mL) | 50 mM (mg) | |
|---|---|---|
| of the solution | NaHCO₃ | APN (mg) |
| 2 | 8.4 | 20 |
| 4 | 16.8 | 40 |
| 8 | 33.6 | 80 |
| 16 | 67.2 | 160 |

### Reaction Between APN and Peptide

The final molar of APN to peptide is selected to be 2. 4 mL of PEG solution will be added dropwise to the 20 mL of peptide solution. The combined PEG and peptide solution is reacted for 2 hours and lyophilized. The desired APN to peptide ratio is 2.

$$\frac{moles of APN}{moles of peptide} = 2$$

The desired ratio is approximately obtained as follows:

$$\frac{2 mL * ( 10 mg \text{/} mL ) / ( 3400 g \text{/} mole )}{10 mL * ( 1 mg \text{/} mL ) / ( 3355.67 g \text{/} mole )} ≅ 2.$$

### Peptide Acrylate Co-Photopolymerization with PEG-Diacrylate

The following describes the peptide acrylate co-photopolymerization with PEG-diacrylate process.

1. The PEG diacrylate precursor solution (20% w/v) with eosin Y as photoinitiator is added directly to the lyophilized acrylated-PEG-peptide and mechanically agitated for one minute.

2. The resulting peptide containing precursor solution is used to encapsulate islets via a selective withdrawal technique.

3. The encapsulating precursor solution is photocrosslinked to form a hydrogel network containing GLP-1.

### Insulin Secretion Test

Three groups of islets are compared by means of a perfusion assay which measures insulin release in response to glucose stimulus. The three groups comprise 10 islets encapsulated with PEG-DA precursor, 10 islets encapsulated with PEG-DA+peptide precursor and 10 naked islets.

In each case, 10 islets (naked or encapsulated) are placed in a flow-through chamber. Upstream glucose concentrations are controlled, and downstream insulin concentrations are measured. The glucose concentration is varied from low (3.3 mM) levels simulating fasting blood glucose levels, to high (16.7 mM) simulating post meal blood glucose levels, and back to low (3.3 mM). The membranes of the Islets are then depolarized by exposure to a 3.3 mM concentration of KC1 to release remaining insulin stored within the islets.

### Characterization of GLP-1 Content in Polymer Conjugate

GLP-1 and its polymer conjugate contain only one tryptophan moiety, the wavelength at 280 nm, at which tryptophan presents characteristic absorption could be used to detect chemical conjugation. The content of GLP-1 in polymer can be calculated from UV spectrophotometry. A series of GLP-1 and PEG-GLP-1 solutions in 0.01 N NaOH are used for calibration curves. The process for characterizing the GLP-1 content in the polymer conjugate is set forth below.

1) Dissolve 0.8 mg (or 1.6 mg) NaOH in 2 mL (4 mL) dH20 to prepare 0.01 N NaOH.

2) Dissolve 2 mg GLP-1 and 2 mg PEG-GLP-1 polymer conjugate separately in 2 ml 0.01 N NaOH solution.

3) Prepare the following concentrations of GLP-1 and PEG-GLP-1 conjugate: 0.01, 0.025, 0.05, 0.075, 0.1, 0.2, 0.6, 1 mg/mL by performing the following steps:
a. Take 0.01 mL (or 10 μL) of solution prepared in step 2, and add 990 μl of 0.01 N NaOH
b. Take 0.025 mL (or 25 μL) of solution prepared in step 2, and add 975 μl of 0.01 N NaOH
c. Take 0.05 mL (or 50 μL) of solution prepared in step 2, and add 950 μl of 0.01 N NaOH
d. Take 0.075 mL (or 75 μL) of solution prepared in step 2, and add 925 μl of 0.01 N NaOH
e. Take 0.1 mL (or 100 μL) of solution prepared in step 2, and add 900 μl of 0.01 N NaOH
f. Take 0.2 mL (or 200 μL) of solution prepared in step 2, and add 800 μl of 0.01 N NaOH
g. Take 0.6 mL (or 600 μL) of solution prepared in step 2, and add 400 μl of 0.01 N NaOH.
   The UV spectra are scanned from 200 to 400 nm to confirm that the optical density at 280 nm (OD280) is sensitive enough to quantify the amount of GLP-1 in the polymer conjugate. Calibration curves illustrate the correlations between the concentrations of GLP-1 or GLP-1 PEG conjugate and OD280. Based on these curves the content of peptide in the polymer conjugate can be calculated.

### Determination of Peptide Content Using μBCA Assay

The peptide content may be determined by performing a μBCA assay. The μBCA assay is a standard procedure for quantifying the amount of peptide/protein in a sample. A standard μBCA assay may be provided in a kit manufactured by Pierce. The μBCA assay may be performed according to the manufacturers recommended protocols.

### Bioactivity comparison of GLP-1 Polymer conjugate and GLP-1

The physiological level of GLP-1 in the body is 5-15 pM, and this increases up to 20-30 pM after meal ingestion. However, for isolated islets insulinotropic activity of GLP-1 against rat pancreatic islets began at 2.5 nM and was saturated at 250 nM.

The comparison experiment requires preparation of a solution media containing a known amount of glucose and GLP-1. Two concentrations of GLP-1, 5.88 nM and 588 nM are prepared.

In order to prepare 588 nM and 5.88 nM GLP-1(7-37) solution:

1) Dissolve 1 mg of peptide in 500 mL solution to obtain a peptide concentration of 588 nM

2) Take 5 mL from 588 nM peptide solution and add 495 mL KRB buffer in order to dilute the concentration to 5.88 nM.

Insulin secretion tests show that encapsulating pancreatic islets in a PEG coat functionalized by the insulinotropic agent GLP-1 can dramatically increase the insulin secretion of the islets in the presence of glucose. The PEG coating can successfully protect the islets in vivo while allowing the diffusion of essential molecules such as oxygen and nutrients through the polymer layer, and the increased insulin secretion can reduce the quantity of islets necessary to reach normoglycemia. Thus, coating pancreatic islets in the manner described herein may prove to be an effective tool in fighting diabetes.

While various embodiments of the invention have been described, it will be apparent to those of ordinary skill in the art that many more embodiments and implementations are possible within the scope of the invention. Accordingly, the invention is not to be restricted except in light of the attached claims and their equivalents.

1. A functionalized coating for encapsulating pancreatic islets comprising:
a thin poly(ethylene glycol) (PEG) hydrogel adapted to coat pancreatic islets, the PEG hydrogel having crosslinked networks of PEG diacrylate; and
an insulinotropic agent incorporated into the crosslinked networks of the PEG diacrylate.

2. The functionalized coating for encapsulating pancreatic islets of claim 1, wherein the insulinotropic agent comprises glucagon-like peptide-1 (GLP-1).

3. The functionalized coating for encapsulating pancreatic islets of claim 2, wherein the amine terminus of the peptide is functionalized with an acrylate moiety.

4. The functionalized coating for encapsulating pancreatic islets of claim 1, wherein the peptide is copolymerized with the PEG diacrylate via photo initiation.

5. The functionalized coating for encapsulating pancreatic islets of claim 1, wherein GLP-1 is incorporated in the PEG hydrogel by a chemical conjugation between acryloyl-PEG-N-hydroxysuccinimide (acryloyl-PEG-NHS) ester.

6. Prepared tissue for implantation into a patient comprising:
donor tissue; and
a coating encapsulating the donor tissue, the coating functionalized with glucagon-like-peptide-1 (GLP-1).

7. The prepared tissue of claim 6, wherein the donor tissue comprises pancreatic islets.

8. The prepared tissue of claim 7 wherein the pancreatic islets comprise human pancreatic islets.

9. The prepared tissue of claim 7 wherein the pancreatic islets comprise porcine pancreatic islets.

10. The prepared tissue of claim 8, wherein the functionalized coating comprises a poly(ethylene glycol) (PEG) hydrogel.

11. The prepared tissue of claim 10, wherein the GLP-1 peptide is incorporated into a crosslinked network of PEG hydrogel by functionalizing an aminee terminus of the peptide with an acrylate moiety.

12. The prepared tissue of claim 11, wherein the GLP-1 peptide is copolymerized with PEG diacrylate via photo initiation.

13. A method of coating biological material for implantation into a patient comprising:
creating a precursor solution that includes a peptide;
adding the biological material to the pre-polymer solution;
encapsulating the biological material by selectively withdrawing the biological material within the precursor solution;
photocrosslinking the precursor solution encapsulated biological material to form a crosslinked hydrogel network incorporating the peptide.

14. The method of claim 13 wherein the pre-polymer solution comprises a poly(ethylene glycol) (PEG) diacrylate pre-polymer solution.

15. The method of claim 13 wherein the biological material comprises pancreatic islets.

16. The method of claim 13 wherein the peptide comprises glucagon-like-peptide-1.

17. The method of claim 13 wherein creating a pre-polymer solution that includes a peptide further includes:
dissolving the peptide in an aqueous sodium bi-carbonate buffer solution;
separately dissolving acrylate-PEG-NHS in an aqueous sodium bi-carbonate buffer solution;
adding the acrylate-PEG-NHS solution to the peptide solution; and
allowing the solutions to react to form a PEG-peptide conjugate.

18. The method of claim 17 wherein the peptide is glucagon-like-peptide-1 (GLP-1).

19. The method of claim 17 further comprising lyophilizing the combined PEG and peptide solutions after they have been allowed to react.

20. The method of claim 19 further comprising separating acrylated and unacrylated peptides from byproducts of the reaction by dialysis against de-ionized water.

21. The method of claim 20 further comprising determining the content of the PEG-peptide conjugate.

22. The method of claim 17 wherein the molar ratio of acrylate-PEG-NHS to peptide is approximately 2.
